(19) United States
(12) Patent Application Publication
(10) Pub. No.: US 2015/0091581 A1
(43) Pub. Date: Apr. 2, 2015

(54) ISFET ARRAY FOR DETECTING A SINGLE NUCLEOTIDE POLYMORPHISM

(71) Applicant: GENE ONYX LIMITED, Tortola (VG)

(72) Inventors: Mohammadreza Sohbati, Surry (GB); Calvin Sim, Hong Kong (CN)

(73) Assignee: GENE ONYX LIMITED, Tortola (VG)

(21) Appl. No.: 14/388,822

(22) PCT Filed: Mar. 19, 2013
(86) PCT No.: PCT/GB2013/050715

§ 371 Date: Sep. 29, 2014

(30) Foreign Application Priority Data
Mar. 30, 2012 (GB) 1205773.3

(51) Int. Cl. G01N 27/26; G01N 33/68

(52) U.S. Cl. CPC G01N 27/26; G01N 33/6803
USPC 324/464

(57) ABSTRACT

Apparatus comprising a module for detecting a single nucleotide polymorphism, SNP, within a genome and comprising: four reaction chambers for receiving a fluid sample, the reaction chambers each containing a different one of the bases A, C, G, T, and a primer; a reference electrode which in use is immersed in said fluid sample; a pair of Ion Sensitive Field Effect Transistors, ISFETs, associated with each reaction chamber, the ISFETs of each pair comprising respective sensing membranes, or a common sensing membrane, exposed within the associated reaction chamber; and a plurality of difference detectors, each difference detector having a pair of inputs coupled respectively to outputs of ISFETs associated with different reaction chambers.

### TECHNICAL FIELD

The present invention relates to efficient polynucleotide sequencing and SNP detection using an ISFET array.

### BACKGROUND

Deoxyribonucleic Acid (DNA) is a long molecule in the nucleus of cells, and which contains genetic “instructions” used in the development and functioning of living organisms. FIG. 1 shows a section of DNA having a double-helical structure, with an outer backbone of sugars and phosphates and an inner part of bases. The information is encoded in the sequence of the bases which are of four types; Adenine, Cytosine, Guanine and Thymine represented with their initials as A, C, G and T respectively. On the double strands, the bases sit in a complementary way by making hydrogen bonds, as shown in FIG. 2, so that Adenine pairs only with Thymine and Cytosine pairs only with Guanine. Segments of DNA are called genes that encode the projection of particular proteins. 99.9% of the code is similar among the human population and it is the remaining 0.1% that makes the differences. Single Nucleotide Polymorphism is a single mutation of a gene that can be found in more than 1% of a population. Finding SNPs provides valuable information both in recognizing the genetic differences in individuals compared to the majority of the population, and also for their subsequent diagnosis.

With advances in genetic science, sequencing the human genome has helped identify the roots of many deficiencies and inherited diseases by discovering a relation between particular polymorphisms of a gene and a disorder. Detection of single nucleotide polymorphisms (SNPs) in an individual genome and mapping it with information on the effect of the SNP on metabolism can improve and facilitate diagnostics.

The double helical structure of DNA, as shown in FIG. 1, can be denatured by increasing the temperature or by using an enzyme. Due to the complementary nature of the DNA strands, it is possible, by designing an appropriate probe/primer and providing the nucleotides, to replicate the molecule by complementary replication of a template/target. In this way, in order to find out whether a particular sequence is in the genome of a person or not, a primer can be designed to interact with denatured single strands of target DNA. If it matches then it can extend on the target [2]. This extension of a primer on the target releases pyrophosphate and H⁺ ions [3]. Therefore, by detecting whether the acidity in the analyte has increased or not, it is possible to determine whether extension has occurred or not; and consequently whether the designed sequence was matched or not.

In recent years it has been a field of interest to provide point-of-care solutions that can perform the necessary tests quickly in such a way that there would not be any need for a laboratory set up and specialist. One such method [1] in this field relies on the use of Ion-sensitive Field-effect Transistors (ISFETs) which are compatible with CMOS (Complementary Metal Oxide Semiconductor) technology and which benefit from its associated advantages. The ISFET is used as a sensor for measuring the pH and for detection of pH drop in order to indicate whether extension has occurred or not.

However, the presently used platform for ISFETs has made them power and memory intensive. The presently used platforms are required to process large amounts of data in order to provide a yes/no answer saying a particular polymorphism is detected in the target DNA or not. These intensive requirements are mainly due to the existence of sources of error that need to be cancelled out in the measurement.

An ISFET is a member of the floating gate FET family and whose gate voltage is provided from a reference electrode (“remote gate”) through an electrolyte and a sensitive membrane on top of its floating gate. FIG. 3 shows a representation of an ISFET. While in the early development stages, ISFETs used different materials for providing such sensitivity to the concentration of ions in the electrolyte, requiring special processing, they are presently implemented in commercial CMOS (Complementary Metal-Oxide-Semiconductor) technology [5], for example as shown in FIG. 4, using the passivation layer (Si3N4 on top of SiO2) as the sensing membrane so that ISFET specific processing steps are not required. The sensing membrane material can bind with H+ ions in the electrolyte, creating a charge surface on top of it and coupling that charge to a metal layer beneath the sensing membrane. It has been shown that the voltage on the sensitive membrane relative to the reference electrode depends on the ion concentration [4]. ISFETs with other sensing membranes are also known, for example instead of Si3N4, it may be formed of Ta2O5.

It is possible to express mathematically the relationship between pH (H+) and voltage to show that the voltage drop from the reference electrode (remote gate G) to the sensing membrane (G′) depends linearly on pH (see equation (1) below). In equation (2) γ represents non-pH related chemical voltages, U_{T} is thermal voltage and a represents the reduction of sensitivity due to the practically non-ideal condition.

V_{G′}=V_{G}−V_{chem} (1)

V_{chem}=γ+2.3αU_{T}pH (2)

A transistor's characteristic is a function of the difference in its gate and source voltages (equation (3) for (a) Weak inversion, (b) Strong Inversion and (c) Velocity Saturation [6]), i.e.

$$\begin{matrix}{I}_{D} = {I}_{D 0} exp \frac{{V}_{G}}{{nU}_{t}} ( exp \frac{- {V}_{s}}{{U}_{t}} - exp \frac{- {V}_{D}}{{U}_{t}} ) & ( 3 a ) \\ {I}_{D} = {μ}_{n} {C}_{ox} \frac{W}{L} [ ( {V}_{GS} - {V}_{T 0} ) {V}_{DS} - \frac{{V}_{DS}^{2}}{2} ] & ( 3 b ) \\ {I}_{D} = \frac{{μ}_{n} {C}_{ox}}{2} \frac{W}{L} {( {V}_{GS} - {V}_{T 0} )}^{2} ( 1 + λ {V}_{DS} ) & ( 3 c )\end{matrix}$$

Accordingly, several interfaces have been designed to provide the required feedback to make all parameters constant such that this voltage difference also remains constant, while their absolute values follow the variation in the chemical voltage drop resulting from pH change (the floating gate and the source of the intrinsic MOSFET (metal-oxide-semiconductor field-effect transistor) follow the pH change). A common interface for this purpose is shown in FIG. 5.

However, ISFETs can suffer from non-ideal behaviour. For example, this can include chemical and thermal noise, and drift, which is degradation of the sensing membrane due to trapped charge (this has been reported as the main causes of its threshold voltage variation [8, 9]). Drift appears as a DC offset, varying over time with change rates of a few millivolts per hour [10][5]. Care must be taken when performing measurements, and, whenever a pH change is measured relative to another pH, the measurement must be calculated in a differential manner using a Reference FET (REFET). [NB. The REFET should not be confused with the reference electrode—or remote gate—of FIG. 3.]

### ISFETs in SNP Detection

The use of an ISFET in sequencing and SNP detection relies on the fact that hydrogen ions are released as a result of hydrolysis of pyrophosphate, a by-product of the extension of primer/probe strands on target DNA strands when they match (i.e. their sequences are complementary)[3], and the increased concentration of these ions on and around the sensing membrane allows the occurrence of the reaction to be detected for some time after the reaction has occurred. In this instance, rather than taking an absolute measurement of the pH, detection of its change is enough to be able to determine whether the designed probe with a known sequence matches with the target. Therefore, having a REFET that is used to detect all non-ideal signals and subsequently cancel them out from a working ISFET in a differential configuration, can provide for a reliable monitoring of the pH change [11, 12]. Consequently, the REFET is exposed to a mixture of non-extendable (i.e. non-matching) primer with nucleotides, enzymes and target DNA strands to provide the same condition as for the working ISFET except the pH change [13]. FIG. 6 shows the configuration that is typically used during ISFET-based sequencing for SNP detection.

### Existing Technology

Current technology uses the configuration discussed above to implement arrays for parallel detection of different SNPs, as represented in FIG. 27.4.1 of reference [1] (D. M. Garner, Hua Bai, P. Georgiou, T. G. Constandinou, S. Reed, L. M. Shepherd, W. Wong, K. T. Lim and C. Toumazou, “A multichannel DNA SoC for rapid point-of-care gene detection,” in Solid-State Circuits Conference Digest of Technical Papers (ISSCC), 2010 IEEE International, 2010, pp. 492-493). To detect the existence of a sequence and subsequently a mutation at a base locus of n+1, a primer of length n is designed and mixed with the target DNA and enzymes. It is inserted into four chambers, each having an ISFET to sense pH. Each chamber contains only one type of nucleotide (i.e. a first chamber contains only nucleotide type A, the second only C, the third only G and the fourth only T). The pH drops (as a result of the extension of the designed primer on the target DNA strand) only in the chamber that contains the nucleotide complementary to that at position n+1, as it is the only one that can extend the primer. All the ISFETs in the four chambers are compared with the REFET as discussed previously. [NB. In a heterozygate case, as opposed to a homozygate case, two different nucleotide types will be complementary, which results in the pH dropping in two different chambers. This is discussed further below.]

ISFET arrays may be designed and constructed to provide multiple sets of (four) ISFET SNP detectors. Such an array may provide for multiple testing for the same SNP, e.g. to provide increased detection for a given SNP, and/or for testing for different SNPs. In the latter case, the wells of each set of ISFETs (for each different SNP test) will contain a different primer. In some cases, these arrays may be extremely large.

This current platform has drawbacks. High power and memory requirements are the main problems that result from the interfaces for overcoming non-ideal ISFET behaviour, and from the way in which the information must be processed in order to cancel out noise and parasitic effects.

FIG. 27.4.2 of [1] shows a system overview of one of the cutting edge System on Chips. As shown in that Figure, for the forty ISFETs in the array (one of which is the REFET) plus ten temperature sensor readouts, fifty sigma-delta modulators are utilised as well as ISFET offset cancelling circuitry. The complexity with this system increases with the number of ISFETs in the array, and the intensive power and memory requirements can easily be seen by the number of ADCs that are present and the control unit. Some of the drawbacks in a system such as that depicted in the Figure in reference [1] are explained in the following paragraphs.

### Small pH Changes and Sensitivity

The extension of nucleotides within the chambers occurs in a buffer solution and the pH drop depends on the mixture of its ingredients (e.g. the amount and density of them defines how much ion would result in the reaction and what its density would be). In practice, the pH typically drops one unit or less. An example of the pH change during extension is shown in the graph of FIG. 7. Considering an ideal sensitivity of 59 mV/pH at room temperature for a perfect sensing membrane, it can be expected that the voltage change on the floating gate after a capacitive coupling is at best in the region of 10-60 mV, and in practice it is less than 59 mV; for instance 44 mV/pH is reported in referenced article [14] (P. Georgiou and C. Toumazou, “ISFET characteristics in CMOS and their application to weak inversion operation”).

### Single REFET

In a large array, the whole chip relies on one REFET, as the conditions for all ISFETs need to be the same. If the REFET behaves erroneously, all the measurements will be biased with an error. Moreover, the REFET will not and indeed cannot be identical to all other ISFETs in terms of its analyte as different SNPs require different primers.

### Reference Electrode

To provide common conditions, a sole reference electrode or remote gate is used for the whole chip and for the REFET. Therefore, making the ISFETs' remote gate bias equi-potential is difficult and requires either challenging micro-fluidic design of an electrolyte (e.g. salt bridges) that conducts the potential equally, or routing and designing stable reference electrodes at different points on the array structure.

### Monitoring/Tracking the Reaction

The known techniques require the monitoring of reactions in order to interpret the behaviour as an indication of pH change and detection of SNPs. This is the case even though the final answer (or output) is a single bit of information indicating whether pH has dropped or not, and consequently whether the SNP has been found or not (one or zero). The reaction must be monitored from the beginning in order to be able to obtain a result.

### ISFET Interface

Using a single communication interface for all ISFETs in an array is not ideal, as it requires the addition of switches at the drains and sources of the ISFETs in order to connect each to the interface (in turn) to monitor reactions. This affects the performance of the ISFETs and requires fast scanning of all the ISFETs through the chip in order to have a substantially continuous measurement of pH across the array. Consequently, considerable effort is required to multiplex and de-multiplex the measurements read from a single interface. Moreover, this results in a varying load for an analogue differential pair, another input of which is the REFET. Therefore, the only way to effectively monitor all ISFETs across an array in parallel, with differential measurement, is to provide a single interface for each ISFET and record its data over time in order to later subtract it from data recorded for the REFET. In this case, either all of the ISFETs must be read simultaneously or the array has to be scanned at high speed to sample each ISFET at time intervals of less than the length of time taken for the chemical reaction to occur [16].

### Analogue to Digital Converters (ADCs)

The known configurations require high precision conversion of the analogue data to digital data, and a processing unit to read the recorded data and subtract the REFET data from the working ISFET data. High precision is important, as a pH change of only a few tens of millivolts at most (ideally 59 mV when pH changes by one unit) must be detected, and for which pH change the quantization noise should not be comparable, especially as the signal is accompanied by drift from the ISFET itself. One proposed design requires 10-bit ADCs for each single ISFET in the array [1]. This architecture demands a high volume of memory as well as a processing unit, resulting not only the design being particularly power and memory intensive, but also greatly increasing the complexity of the chip design. With this architecture, a large amount of data must be collected in order to monitor and interpret the detection of a pH drop—namely 40 bits of information from four ISFETs, each monitoring the possibility of extension of the primer with each of the four base types (A, C, G and T), in addition to 10 bits for the REFET, makes 50 bits of information per sampling time for each SNP. This of course scales up with the number of SNPs to be analyzed in parallel.

The major disadvantages and problems with current set-ups for ISFET-based SNP detection have been discussed above. Problems such as dependency on performance of a single REFET, design of reference electrode and demand for high precision ADCs, monitoring the reaction from the beginning, and collection of large amounts of redundant data resulting in high power and memory consumption, have been described. The root of the aforementioned problems is in the analogue nature of the known platforms, as well as the algorithm required to eliminate anomalous signals after measurement and the non-ideal behaviour of the ISFET.

Similar considerations apply in the case of SNP detection in RNA.

### SUMMARY

According to a first aspect of the invention, there is provided an apparatus comprising a module for detecting a single nucleotide polymorphism, SNP, within a genome and comprising:
four reaction chambers for receiving a fluid sample, the reaction chambers each containing a different one of the bases A, C, G, T, and a primer;
a reference electrode which in use is immersed in said fluid sample;
a pair of Ion Sensitive Field Effect Transistors, ISFETs, associated with each reaction chamber, the ISFETs of each pair comprising respective sensing membranes, or a common sensing membrane, exposed within the associated reaction chamber; and
a plurality of difference detectors, each difference detector having a pair of inputs coupled respectively to outputs of ISFETs associated with different reaction chambers.

Said plurality of difference detectors may have pairs of inputs coupled to outputs of different pairs of reaction chambers.

The apparatus may comprise four difference detectors X, Y, Z and W, these having inputs coupled to reaction chamber ISFETs as follows:
X: A and T
Y: T and C
Z: C and G
W: A and G.

The apparatus may comprise an output detector for monitoring the outputs provided by the difference detectors in order to detect output states indicative of nucleotide additions that are not possible.

The apparatus may comprise a processor coupled to said output detector and configured to react to detection of a not possible state by performing an adjustment of the apparatus to eliminate said not possible state.

Said adjustment being made to the offset of the plurality of difference detectors.

Said processor may be configured to perform said adjustment by one or a combination of: altering a bias voltage applied to said reference electrode, altering the difference detector bias current, altering a bias reference current, and altering the current through a buffer stage.

The difference detectors may be differential amplifiers, may be trans-conductance amplifiers, and may be comparators.

The apparatus may comprise a plurality of said modules configured to detect different SNPs.

According to a second aspect of the invention there is provided a method of detecting a Single Nucleotide Polymorphism, SNP, within a genome, the method comprising:
receiving a fluid sample in four reaction chambers, the reaction chambers each containing a different one of the bases A, C, G, T, and a primer;
detecting any pH changes within the reaction chambers using a pair of Ion Sensitive Field Effect Transistors, ISFETs, associated with each reaction chamber, the ISFETs of each pair comprising respective sensing membranes, or a common sensing membrane, exposed within the associated reaction chamber; and
providing an output from a plurality of difference detectors, each difference detector having a pair of inputs coupled respectively to outputs of the ISFETs associated with different reaction chambers.

The method may further comprise monitoring the outputs provided by the difference detectors in order to detect output states indicative of nucleotide additions that are not possible.

Monitoring the outputs provided by the difference detectors may comprise looking up the outputs in a truth table.

The method may further comprise reacting to detection of a not possible state by performing an adjustment of the apparatus to eliminate said not possible state.

Said adjustment may be made to the offset of the plurality of difference detectors.

Performing an adjustment of the apparatus may comprise one or a combination of: altering a bias voltage applied to a reference electrode, altering the difference detector bias current, altering a bias reference current, and altering the current through a buffer stage.

The difference detectors may be differential amplifiers, may be trans-conductance amplifiers, and may be comparators.

According to a third aspect of the invention there is provided an apparatus comprising a module for detecting a single nucleotide polymorphism, SNP, within a genome and comprising:
four reaction chambers for receiving a fluid sample, the reaction chambers each containing a different one of the bases A, C, G, T, and a primer;
a reference electrode which in use is immersed in said fluid sample; and associated with each reaction chamber
   a pair of Ion Sensitive Field Effect Transistors, ISFETs, the ISFETs of each pair comprising respective sensing membranes, or a common sensing membrane, exposed within the associated reaction chamber, and the pair of ISFETs being coupled in a push-pull configuration;
   a MOSFET inverter stage having an input coupled to an output of said push-pull configuration; and
   an output stage providing a digital output that is switchable in dependence upon the voltage present at said sensing membrane(s).

Said MOSFET inverter stage may comprise one MOSFET inverter, or a number of stacked MOSFET inverters.

The apparatus may comprise a reference chamber, the reference chamber containing a non-matching primer.

The apparatus may comprise a reference output detector for monitoring the output of the reference chamber in order to detect erroneous deviation within the apparatus.

The apparatus may further comprise a processor coupled to said reference output detector and configured to react to detection of erroneous deviation by performing an adjustment of the apparatus to eliminate said deviation.

The apparatus may further comprise an output detector for monitoring the digital outputs of the output stage in order to detect output states indicative of nucleotide additions that are not possible.

The apparatus may comprise a processor coupled to said output detector and configured to react to detection of a not possible state by performing an adjustment of the apparatus to eliminate said not possible state.

Said processor may be configured to perform said adjustment by altering a bias voltage applied to said reference electrode.

Said processor may be configured to perform said adjustment by altering a programmable inverter to adjust a switching threshold.

The apparatus may comprise a plurality of said modules configured to detect different SNPs.

According to a fourth aspect of the invention there is provided a method of detecting a Single Nucleotide Polymorphism, SNP, within a genome, the method comprising:
receiving a fluid sample in four reaction chambers, the reaction chambers each containing a different one of the bases A, C, G, T, and a primer;
detecting any pH changes within the reaction chambers using a pair of Ion Sensitive Field Effect Transistors, ISFETs, associated with each reaction chamber, the ISFETs of each pair comprising respective sensing membranes, or a common sensing membrane, exposed within the associated reaction chamber, and the pair of ISFETs being coupled in a push-pull configuration;
coupling the output of the push-pull ISFETs to the input of a MOSFET inverter stage; and
providing a digital output from an output stage, the digital output being switchable in dependence upon the voltage present at said sensing membrane(s).

The method may comprise also performing the steps of the method on a reference chamber, the reference chamber containing a non-matching primer.

The method may comprise monitoring the output of the reference chamber in order to detect erroneous deviation within the apparatus.

The method may comprise reacting to the detection of erroneous deviation by performing an adjustment of the apparatus to eliminate said deviation.

The method may comprise monitoring the digital outputs of the output stage in order to detect output states indicative of nucleotide additions that are not possible.

Monitoring the digital outputs provided by the output stage may comprise looking up the outputs in a truth table.

The method may comprise reacting to the detection of a not possible state by performing an adjustment of the apparatus to eliminate said not possible state.

Performing said adjustment may comprise altering a bias voltage applied to a reference electrode.

Performing said adjustment may comprise altering a programmable inverter to adjust a switching threshold.

### BRIEF DESCRIPTION OF THE DRAWINGS

FIG. 1 shows a section of DNA, and the complementary base pairs;

FIG. 2 shows the chemical structure of the base pairs;

FIG. 3 is an ISFET;

FIG. 4 is an ISFET in unmodified CMOS technology;

FIG. 5 is a Common ISFET interface, drain-source follower.

FIG. 6 is a configuration used for applying ISFET for SNP detection;

FIG. 7 is a graph showing the pH drop as a result of primer extension;

FIG. 8 shows a sequence from DNA building up to a Chromosome;

FIG. 9 is a REFET-less differential measurement configuration;

FIG. 10 shows memory addressing in an array;

FIG. 11 is an ISFET operation capacitive model;

FIG. 12 is an ISFET capacitive model;

FIG. 13 is an ISFET-based inverter;

FIG. 14 is a chemical switch;

FIG. 15 is a graph comparing behaviour in a chemical switch and a MOSFET inverter when the gate voltage is swept from 0 to supply (3.3V) over 3.3 s;

FIG. 16 is a graph showing ISFET-based Inverter characteristics for different pHs;

FIG. 17 is a single DNAGA pixel configuration;

FIG. 18 is a graph showing a simulation of the pixel operation when pH changes over time;

FIG. 19 is a DNAGA configuration sample;

FIG. 20 is a DNAGA datapath overview;

FIG. 21 is a DNAGA Calibration and Control Algorithm;

FIG. 22 is a Sample State Machine for DNAGA;

FIG. 23 is a REFET-less DNAGA Calibration and Reading Algorithm;

FIG. 24 is a sample REFET-less DNAGA Architecture;

FIG. 25 is a sample Implementation of the combinational logic for detection of invalid states in REFET-less DNAGA;

FIG. 26 is a sample Carnot-map for error detection logic;

FIG. 27 is a sample scenario of adjustment and detection of SNPs for a heterozygote case;

FIG. 28 is an inverter stack configuration with the second inverter being programmable for adjustment of switching point;

FIG. 29 is a REFET-less DNAGA calibration and reading algorithm when a programmable gate inverter is used; and

FIG. 30 shows a block diagram that depicts a differential measurement taken between inputs from two different ISFETs.

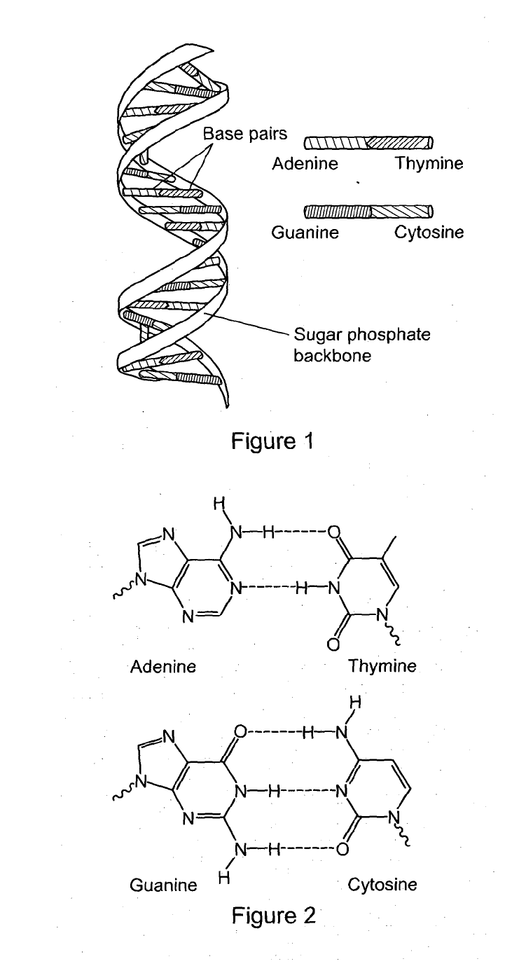

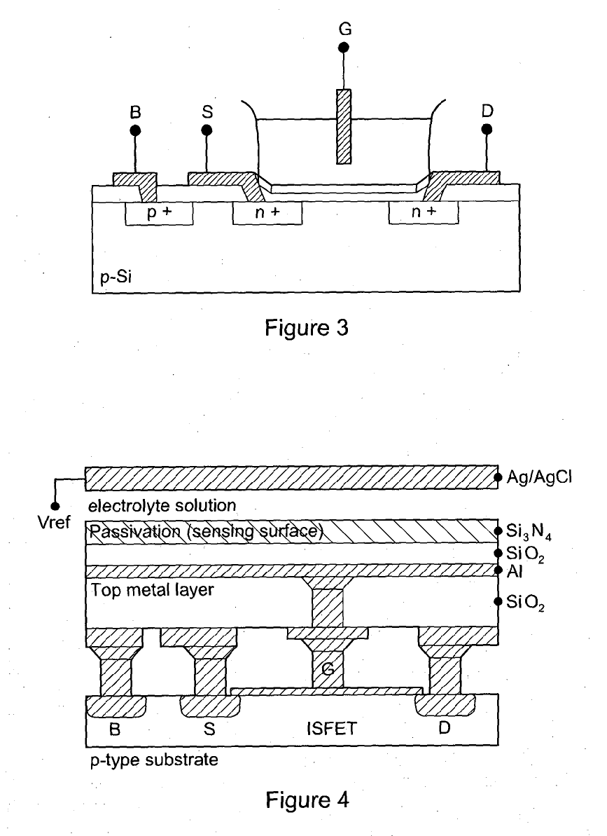

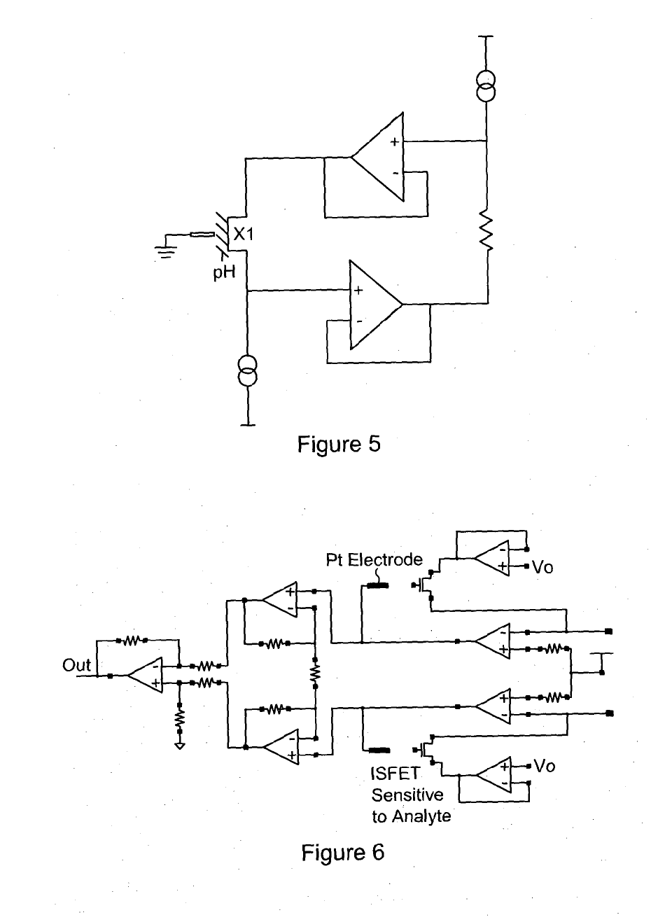

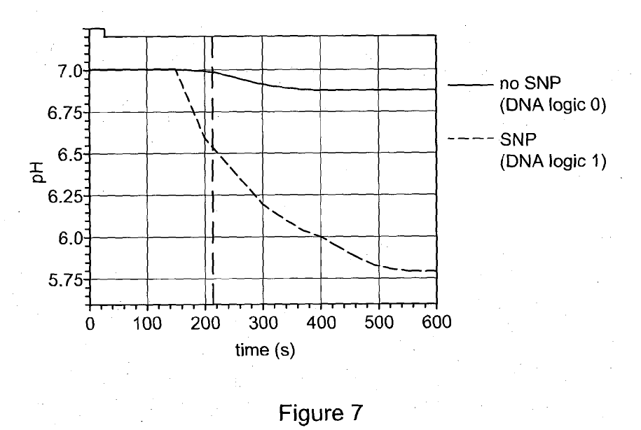

### DETAILED DESCRIPTION

As previously discussed, there are a number of disadvantages and problems with current set ups for ISFET-based SNP detection. Novel methods and apparatuses will now be described that can simplify the operation of such devices dramatically. For example, a new method of differential measurement will now be proposed that not only increases the reliability of pH change measurement, but also reduces the amount of data required to be recorded. A further new method will also be described that provides a direct digital result while also overcoming the inherent issues of ISFET/chemical measurements. However, before moving on the proposed configurations, an error indication idea is explained to clarify the performance of the proposed designs.

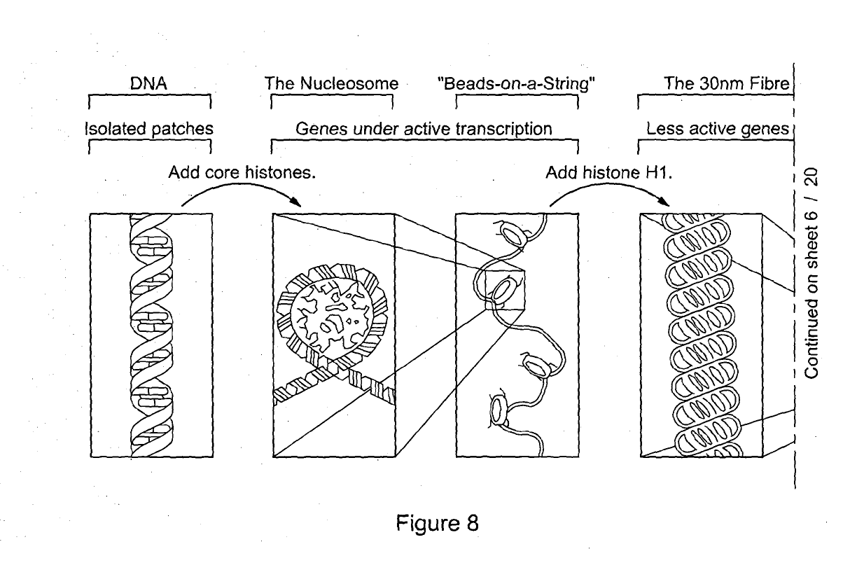

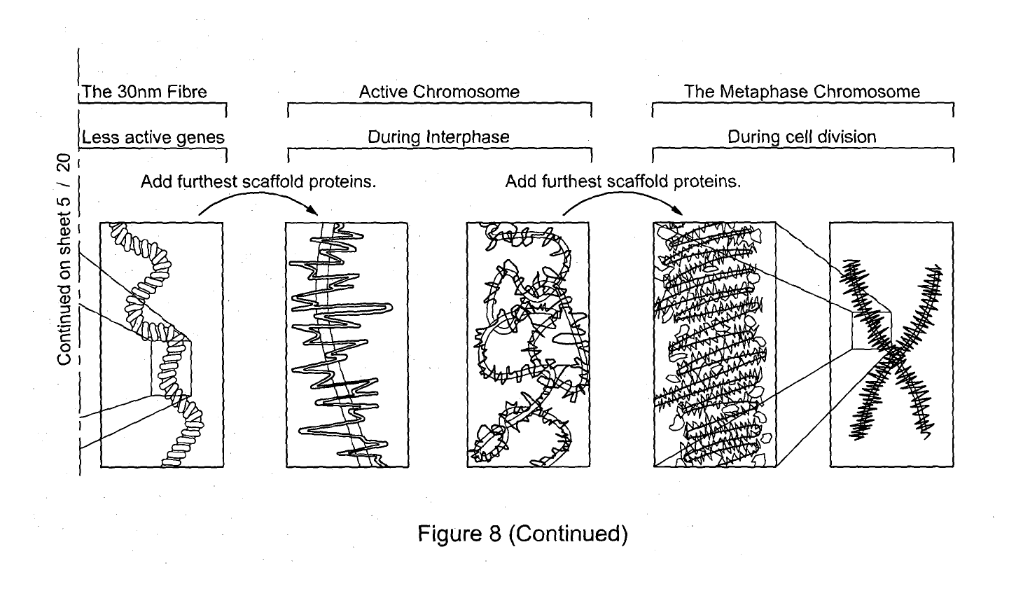

As shown in FIG. 8, DNA molecules are located within pairs of chromosomes. A gene (as a segment of DNA), encoding the projection of a certain protein, sits on a specific part of a particular chromosome pair. The gene sequence can be the same on the two chromosomes of the pair (homozygous) or can be different (heterozygous). When searching for a specific mutation at a particular base position of a gene in an individual, it is possible to find a similar mutation on both chromosome arms (i.e. the individual is homozygote) or two different bases (the individual is heterozygote) located at the point of interest.

For example, for a human being that has 23 pairs of chromosomes, the base in position n of a gene sequence can be different and cause the projection of the gene to be different as a consequence. If the sequence of bases before position n is represented by a and the sequence after that with β, the person that the target gene polymorphism belongs to is homozygote with type A if only a sequence of αAβ exists in his/her genome, so that both arms of the chromosome pair have got αAβ. He/she is heterozygote if two alleles (i.e. different forms of a gene), for example αAβ and αCβ, are found so that one arm of the chromosome pair contains a sequence of αAβ and the other one contains αCβ.

Therefore, potentially, for a single mutation of a gene, there is a space of 4 nucleotides and consequently 10 probable alleles for the individual: four homozygote conditions, when only one of the four bases fills the location in both arms of chromosome pair (αAβ, αCβ, αGβ and αTβ), and six heterozygote cases when two different alleles are found at the location in both arms of the chromosome pair (αAβ and αCβ, αAβ and αGβ, αAβ and αTβ, αCβ and αGβ, αCβ and αTβ, αGβ and αTβ). Therefore, it is not possible that 3 or 4 alleles be found in a person's genome and it cannot have a gap either. Mathematically looking at the problem in an individual,

$$\begin{matrix}Valid Conditions \text{:} homozygous ( \begin{matrix}4 \\ 1\end{matrix} ) + heterozygous ( \begin{matrix}4 \\ 2\end{matrix} ) = 10 & ( 3 ) \\ Invalid Conditions \text{:} ( \begin{matrix}4 \\ 0\end{matrix} ) + ( \begin{matrix}4 \\ 3\end{matrix} ) + ( \begin{matrix}4 \\ 4\end{matrix} ) = 6 & ( 4 )\end{matrix}$$

Looking back to the SNP test, in order to find the polymorphisms of a specific gene in a target DNA of a person, a primer with a complementary sequence to the target gene is designed with length of n in order to find the base in position n+1 in the target DNA strands (the target is taken from a sample of individual's DNA, for example taken from the saliva, and has got both types of DNA on the two arms of chromosomes). The primer and target DNA molecules as well as the required enzymes are inserted into four chambers. Each of the chambers only has one of the four nucleotides (A, C, G or T). In each chamber there is an ISFET measuring the H+ ion concentration. The ISFET located in the chamber that contains the nucleotide which matches with the target and manages to extend the primer, will detect a pH drop. So, from each ISFET/chamber only one bit of information is sought (i.e. a 1 if pH drops, or 0 if not). Out of the four ISFETs, we potentially can get 2⁴=16 conditions. But from the previous paragraph we know that only 10 situations are valid and we expect one of those ten conditions to be found:

Valid results: Homozygote {1000, 0100, 0010, 0001}Heterozygote {1100, 1010, 1001, 0110, 0101, 0011} (5)

Invalid Results: {0000, 1110, 1101, 1011, 0111, 1111} (6)

However, due to the existence of the previously described non-ideal signals (e.g. chemical and thermal noise, as well as drift in the threshold voltage of the ISFET over time) the measurement can be biased with an error. These unwanted signals will have a DC or very low frequency nature and appear as an offset. This error may affect the ISFET output and causes it to behave in a way that could signal a wrong pH drop or cause it to not show a pH drop that has occurred. These noises are common and appear may give rise to one of the 6 invalid conditions; for example when none of the ISFETs report a pH drop, or all of them, or three of them.

### New Differential Scheme (REFET-Less)

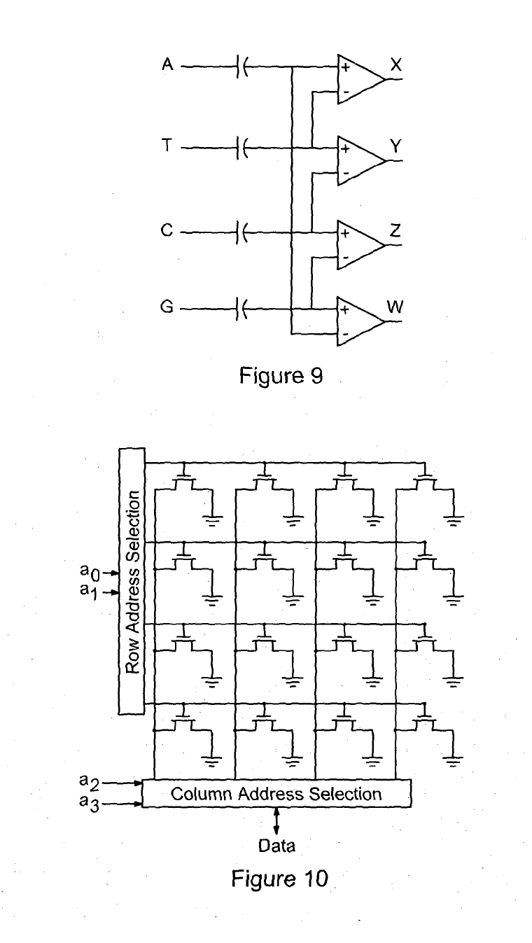

A new architecture will now be described in which there is no longer a requirement for a REFET. Instead of measuring each ISFET signal, a differential measurement is calculated while the signal is also amplified at the point of measurement. This can ease the work of an ADC, but more advantageously an ADC may not even be required. FIG. 9 shows the configuration required for a “REFET-less” differential measurement (as the ISFETs work as floating gate transistors having their signal capacitive coupled, the chambers are represented with capacitors). In this configuration, each chamber (A, T, C, G) has two ISFETs (or indeed two intrinsic floating gate MOSFETs sharing the same sensing membrane). Each ISFET provides one of the input pair transistors in a differential amplifier. Four differential amplifiers are used (X, Y, Z, W). The ISFETs are wired in a way that one of the two ISFETs of a chamber makes the non-inverting input of an amplifier and the other one creates the inverting one in another amplifier. In this configuration, the previously known design of an interface with several buffers and offset-cancelling circuits has been replaced with single simple differential amplifiers.

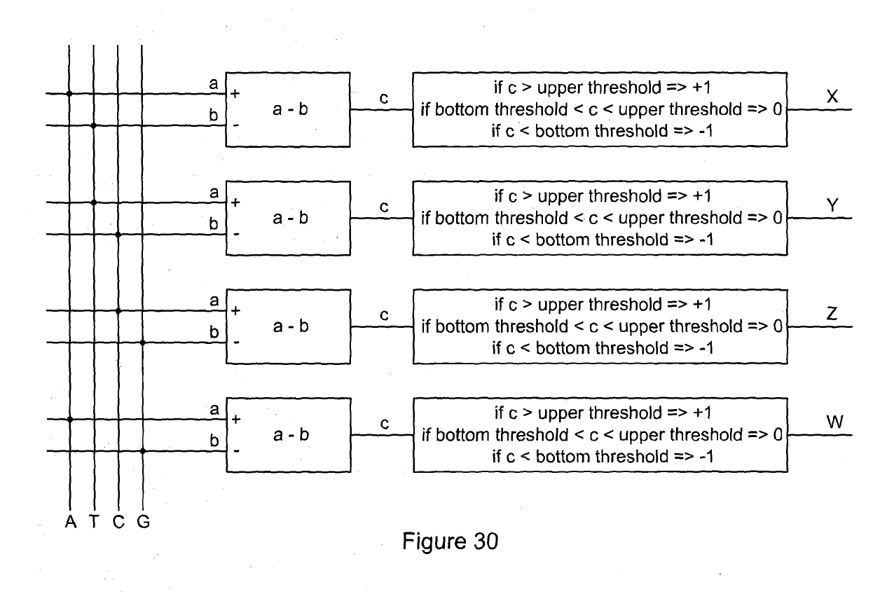

FIG. 30 shows a block diagram that depicts the differential measurement that is taken between inputs (a and b) from two different ISFETs (A, T, C and G). The inputs are differentially measured, and an output c is provided. As shown in FIG. 30 there can be a logic step that determines which of a number of threshold bands the differential output c meets. For example in FIG. 30, there is an upper threshold and a bottom threshold. If c is greater than the upper threshold then the corresponding output X, Y, Z or W will be +1, if c is lower than the bottom threshold then the corresponding output X, Y, Z or W will be −1, or is c falls between the upper and the bottom threshold then the corresponding output X, Y, Z or W will be 0.

The output of the differential amplifiers X, Y, Z and W can be divided into three regions. One is represented by −1 as a result of pH drop on the inverting input ISFET; another with 1 in result of pH drop in the non-inverting one; and 0 for the case when a pH drop has not been detected in either of the two, or has been detected in both of them but has been cancelled out. Considering the 16 conditions (10 valid, and 6 invalid) that can happen in each four chambers, a truth table is created stating which results are valid and what each valid result indicates, and also which ones are invalid (i.e. the output states of the differential amplifiers are indicative of nucleotide additions that are not possible) and require the biasing of the apparatus to be adjusted. Table 1 is an example of the truth table corresponding to the configuration of FIG. 9, where “AR” stands for “Adjustment Required”.

TABLE 1

| A T C G | X Y Z W | Result |
|---|---|---|
| 0 0 0 0 | 0 0 0 0 | AR |
| 0 0 0 1 | 0 0 +1 −1 | G Type |
| 0 0 1 0 | 0 +1 −1 0 | C Type |
| 0 0 1 1 | 0 +1 0 −1 | CG Type |
| 0 1 0 0 | +1 −1 0 0 | T Type |
| 0 1 0 1 | +1 −1 +1 −1 | TG Type |
| 0 1 1 0 | +1 0 −1 0 | TC Type |
| 0 1 1 1 | +1 0 0 −1 | AR |
| 1 0 0 0 | −1 0 0 + 1 | A Type |
| 1 0 0 1 | −1 0 +1 0 | AG Type |
| 1 0 1 0 | −1 +1 −1 +1 | AC Type |
| 1 0 1 1 | −1 +1 0 0 | AR |
| 1 1 0 0 | 0 −1 0 +1 | AT Type |
| 1 1 0 1 | 0 −1 +1 0 | AR |
| 1 1 1 0 | 0 0 −1 +1 | AR |
| 1 1 1 1 | 0 0 0 0 | AR |

In this method, and with this configuration, there is no need for a separate REFET as the common non-ideal signals are cancelled out by the differential measurements. The conditions for the comparison of the measurements are now the same and the only difference is the extending nucleotide and the resulting pH change. Even more advantageously, even for the case that the nucleotide does not match, a release of hydrogen ions can occur as a result of the hybridization of the primer with part of the target DNA, though not to as great an extent. This would not be sensed as an error in the previously used configuration with a separate REFET, but now, with this configuration, even this misleading event is removed.

Beyond the differential measurement that cancels out the non-ideal behaviours, amplification of the differentially measured signal takes place at the point of differential measurement rather than buffering it in the conventional read-out circuitry and then digitizing it for recording. This is particularly advantageous as each signal is amplified equivalently and the burden on the ADC (if indeed it is even still required) is much less.

Where a “not possible” output state is returned, the apparatus can be adjusted to “eliminate” the not possible state, i.e. to correct the effects of drift in the biasing. For those results whereby “Adjustment Required” is returned, the type of adjustment intended are ways of calibration that cancel out any drift in the biasing that appears as an offset. With this configuration, there are several points that can be adjusted or tuned, for example the current source of the differential pair, a bias reference current, the voltage of the reference electrode or the biasing of a buffer stage.

Another further advantage of this configuration is that the design of reference electrode can be simplified. The reference electrode in this configuration is not required to be the same for the whole array, but instead it is enough to just be the same in each set of the four chambers targeting the same gene SNP.

Furthermore, the chip design is made easier and scalability is increased. The output signal is already differential, so sampling and scanning time requirements are less onerous. Also, the signal is already amplified. It is now possible to multiplex the measurements as there is no need to continuously record the data. Therefore, instead of routing four 10 bit buses for each SNP detector, only four wires are now required.

An embodiment has been described that uses differential amplifiers to provide the differential measurement. However, other components can be used in place of differential amplifiers. For example, rather than take the difference in voltages (as is the case for differential amplifiers), it is possible to transform the signal into currents and then subtract currents from each other. This is carried out using trans-conductance amplifiers in place of the differential amplifiers. A further alternative is to use comparators in place of the differential amplifiers. The term “difference detectors” can be used to cover all suitable components. Difference detectors can be seen as “subtractors”, which substract one input from another to find the difference between the two.

It will be appreciated by the person of skill in the art that there are a number of ways in which the differential measurement can be carried out, and that any of them could be used without departing from the scope of the present invention.

### A DNA Gate Array

A novel concept and architecture, referred to here as a DNA Gate Array (DNAGA), for direct digital measurement will now be described. The object here is to obtain only those bits of information required to detect the SNP of interest. Indeed, it is preferential to only look for the pH change once or twice without having to monitor the pH value, and also to return a simple 1-bit answer as to whether the pH in a chamber has dropped or not (1 or 0). In other words, an architecture is proposed in which each ISFET (or pixel, or chamber in general) can be addressed and its response read with a single bit. This is similar to the way memory is read as depicted in FIG. 10.

The reason for designing a DNAGA is that the final information such SNP tests provide is summarized in two bits of information identifying which polymorphism is found in the target DNA strands. This information is then processed to indicate whether it is a homozygous type or a heterozygous one, a wild type or a mutant type, and then the “diagnosis” is given. Such logic cannot be made by single ISFETs for the reasons discussed here in addition to the non-ideal behaviours of ISFETs previously discussed. However, in the DNAGA concept, the unwanted signals are cancelled out (as will be described below) and now the extension of DNA strands provides the required digital information directly at the output of the DNAGA and subsequently at the input gates of a processing unit that may be placed at a later stage in the chip.

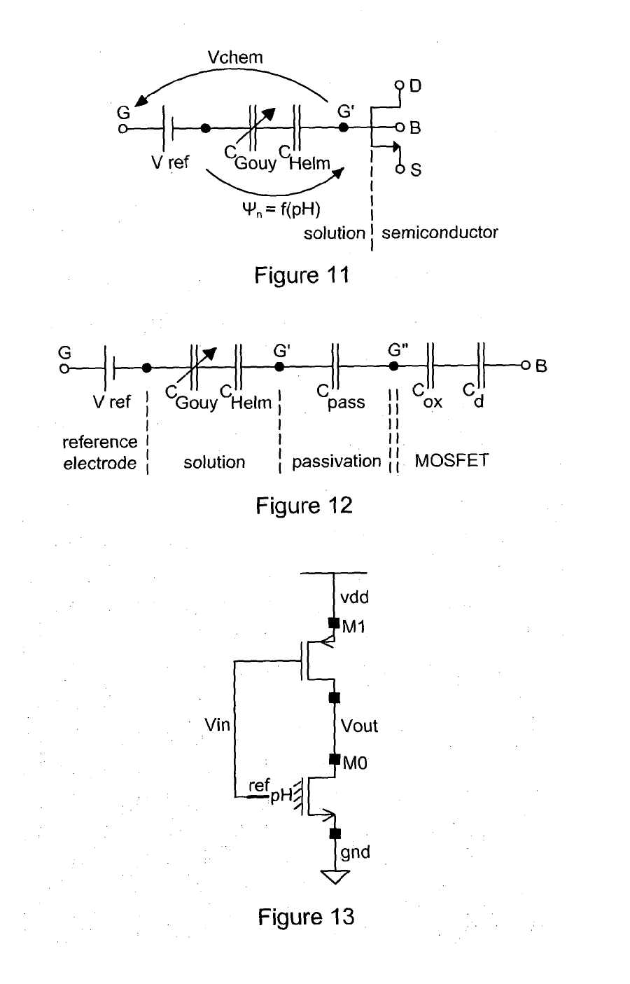

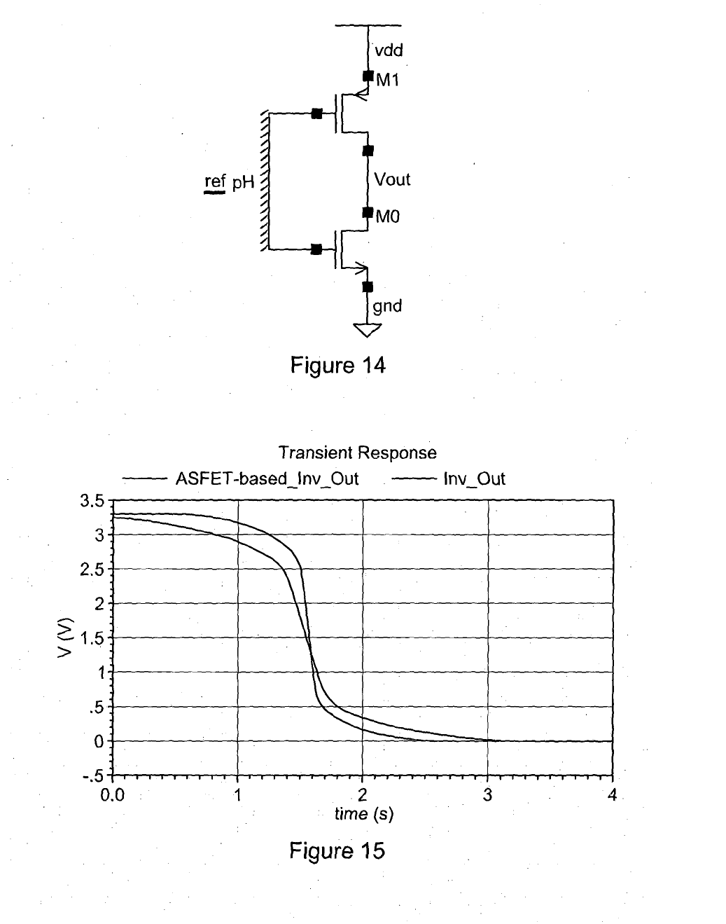

A single ISFET cannot be used in an array configuration, in a digital manner. The ISFET is not able to drive its output when its drain is connected to the drain of other ISFETs in a column and driven by a pull-up PMOS, as the signal driving the ISFET is too small. As previously mentioned, the sensitivity of an ISFET is much less than the ideal 59 mV/pH and the pH change in the buffer solution will, in practice, be less than 1 pH. The transistor is also biased at a mid-operating point, as are the rest of the ISFETs sharing their drains. Another reason for reduction of the sensitivity is the capacitive structure of ISFET [9], as shown in FIGS. 11 and 12.

When the bulk port of the transistor is grounded (constant), the voltage on the floating gate is a fraction of the potential on the sensing membrane. In the following equation (7), n is the slope factor, Cₒₓ is gate oxide capacitance and Cₚₐₛₛ is the capacitance of the sensing membrane (the passivation layer), all summarized in the term ζ. This parameter is defined by the ratio of the sensing membrane area to the area of the floating gate. To make ζ small for a high sensitivity, the ratio of the sensing membrane area to the area of the floating gate must be large. For example, in 0.35 μm technology, in order to have a ζ of 0.1, the sensing membrane area must be more than 500 times larger than the area of the intrinsic MOSFET beneath the sensing membrane.

$${V}_{{G}^{″}} = \frac{{V}_{{G}^{′}}}{1 + \frac{n - 1}{n} \frac{{C}_{ox}}{{C}_{pass}}} = \frac{1}{1 + ζ} {V}_{{G}^{′}} \qquad ( 7 )$$

In the capacitive stack model depicted, the smallest value is defined by the passivation and oxide capacitances, which are very small. For instance in a 0.35 μm technology, they are 0.023 fF/μm² and 4.6 μF/μm². This is much smaller than the fan-out capacitance seen from the drain of the ISFET, which is the sum of all the drain-gate capacitances of the other ISFETs and the pull-up PMOS (each is tens of fF). Therefore, a single ISFET cannot drive such a load and cannot work in such a structure.

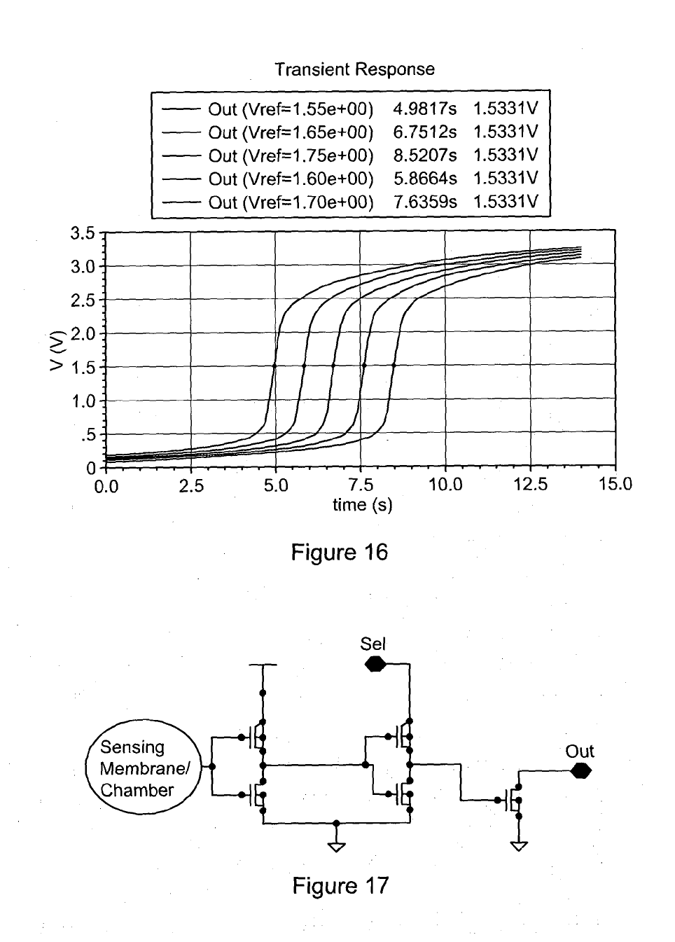

There has been an effort to implement the ISFET in an inverter configuration and also use it as a chemical switch. However, this is rejected because of the weakness discussed in the previous paragraph. The capacitive division of the driving voltage does not let it have a characteristic like an ordinary inverter. Two of the proposed architectures are shown in FIGS. 13 (an ISFET-based inverter) and 14 (Chemical switch), and the graphs shown in FIGS. 15 and 16 which indicate what the outputs of each would look like. In the graph of FIG. 15, chemical switch behaviour is compared with an ideal inverter. The gate voltage was swept from 0 to supply (3.3V) over 3.3 s. The graph of FIG. 16 shows ISFET-based Inverter characteristics for different pHs. Discrimination of the switching point depends on the sensitivity and they can overlap in a range providing neither an exact one nor zero.

Although making the sensing membrane very big relative to the gate area suppresses the effect of ζ, the pH change is not big enough to discriminate between the states. Moreover, the fan-out to fan-in capacitance issue is not resolved with these configurations. They are not digital at all.

Therefore, the proposed solution is to leave the array reading structure of memory as it is, but to provide a powerful digital force driving the output, which later drives the logic gates. So, simply, the ISFET is configured as a push-pull amplifier just for amplifying the signals it senses at the point of measurement and then it is cascaded with a MOSFET (metal-oxide-semiconductor field-effect transistor) inverter such that its output is a proper digital 1 or 0 and its input is a smaller load for the ISFETs. Moreover, as the reaction is not reversible, and the increased concentration of H⁺ ions remains after the extension/hybridization, the occurrence of the reaction can be detected for some time after the reaction has occurred. Thus, whenever the pixel is not needed to be read, its power can be cut, which also can be used for addressing the rows in an array. In fact, the number of stacking MOSFET inverters depends on the technology and designer—the goal is to amplify the signal in such a way that the output of the pixel is vdd or gnd (digital 1 or 0), such that it can properly drive the gates.

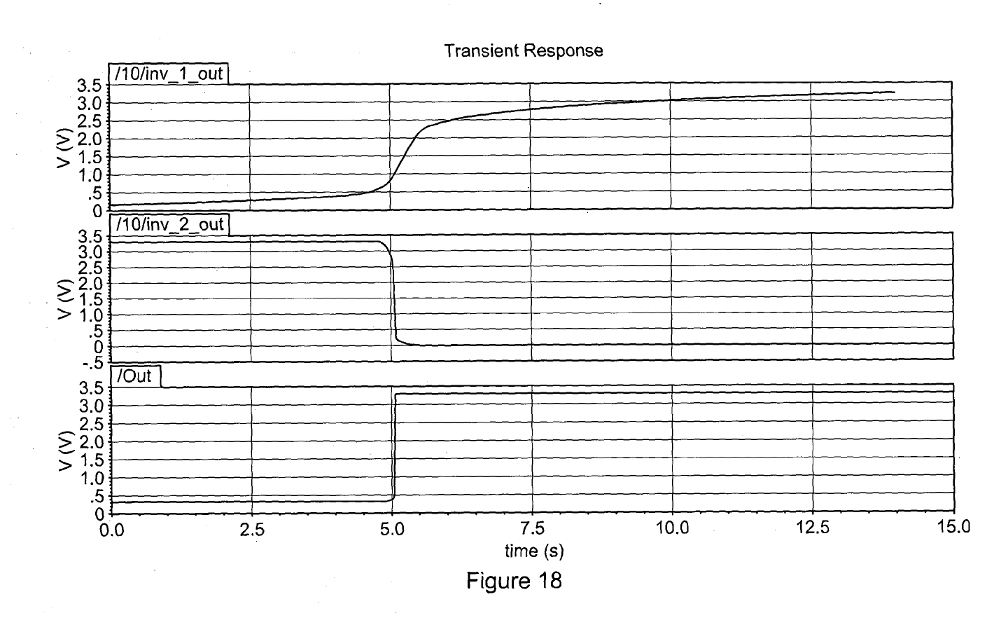

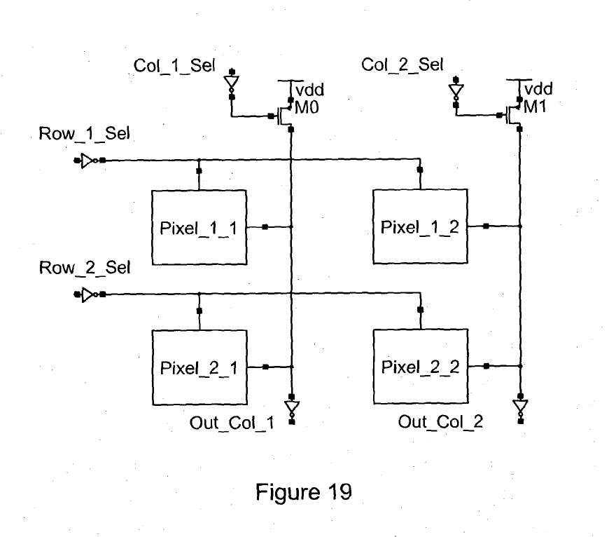

FIG. 17 shows a single DNAGA pixel configuration according to an embodiment. A pair of ISFETs connected to the sensing membrane are configured as a single push/pull amplifier. This, in turn, is cascaded with a MOSFET inverter. The inverter/pixel is joined to the supply through “Sel”, which is connected to the supply when the pixel is selected. FIG. 18 shows three graphs produced by a simulation of the pixel operation when pH changes over time. The first (top) graph is the output voltage of the ISFET-based inverter, the second (middle) graph is the output of the MOS inverter and the last (bottom) graph is the output when the pixel is selected, and it can be seen that this final output is digital. The switching region and point can be set by biasing the reference electrode. Consequently, using this configuration, the output from each chamber/pixel is a single bit that can be either 0 or 1. Having digitized what is read from the pixels/chambers, the pixel can be implemented in an array form, an example of which is shown in FIG. 19 which shows the architecture of a DNAGA configuration sample.

A question that arises is what happens to the non-ideal behaviours that make the measurements unreliable, causing deviation from the correct point and in this case forcing a wrong switching of the inverters. These unwanted signals and behaviours are common among the pixels in all the measurements. Previously, a REFET was used to sense the same signals, except for the pH change, as the primer in it was intentionally non-matching. That REFET concept can be used in the proposed DNAGA, but in a digital way, in the form of a Reference pixel.

When the target DNA strand and the primer match and the primer in the chamber containing the next matching nucleotide extends, the pH drops. The pH drop reduces the voltage drop from the reference electrode to the surface of the sensing membrane (equation 2) so that the potential on the sensing membrane, and consequently the floating gate, increases. In the push-pull/inverter stack configuration, this change makes the output voltage of the ISFET-based push-pull amplifier drop and forces the second inverter to generate a digital 1.

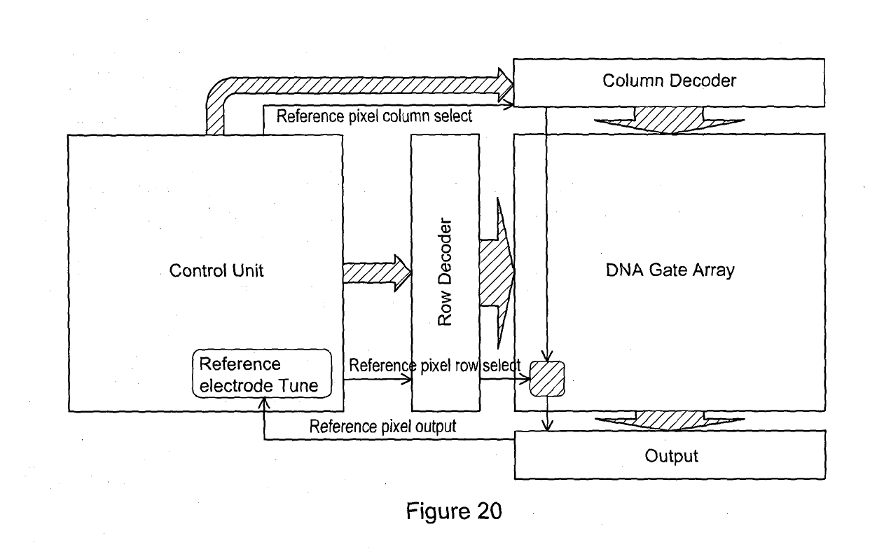

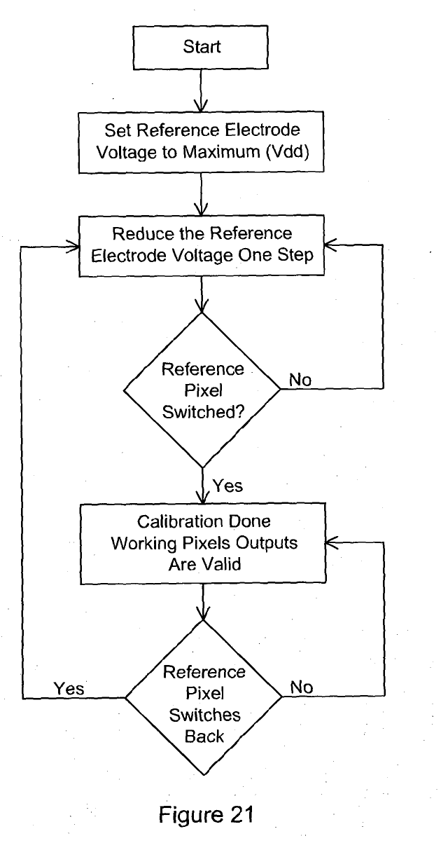

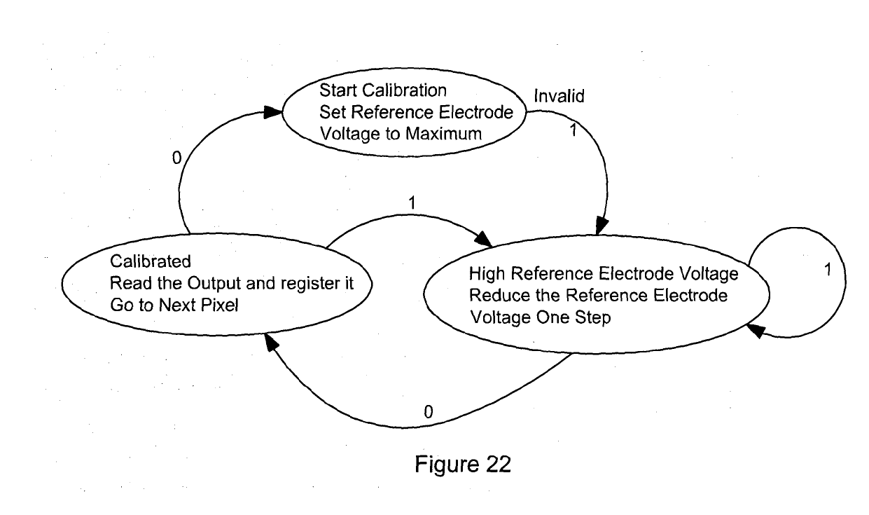

In the scenario where an erroneous deviation occurs (for example due to thermal noise), it may switch the working pixel output from zero to one, but it would also do the same in the Reference pixel and cause that to switch too. This is similar to the case that the reference electrode voltage has increased and caused the switching. So, to cancel out the new added offset, reducing the reference electrode voltage can make the Reference pixel return to its normal state. Consequently any incorrect change in the working pixels is reversed. Such a procedure can be used to calibrate the whole array. FIG. 20 shows an overview of the relation among the working and reference pixels within the architecture, as an overview of the DNAGA data path. FIG. 21 is a flow diagram that outlines the process (or algorithm) of calibrating and controlling the DNAGA by adjusting the voltage of the reference electrode. FIG. 22 is a sample state machine for DNAGA. It can be seen from FIG. 22 that an invalid signal from the reference pixel changes the state.

### A REFET-Less DNAGA

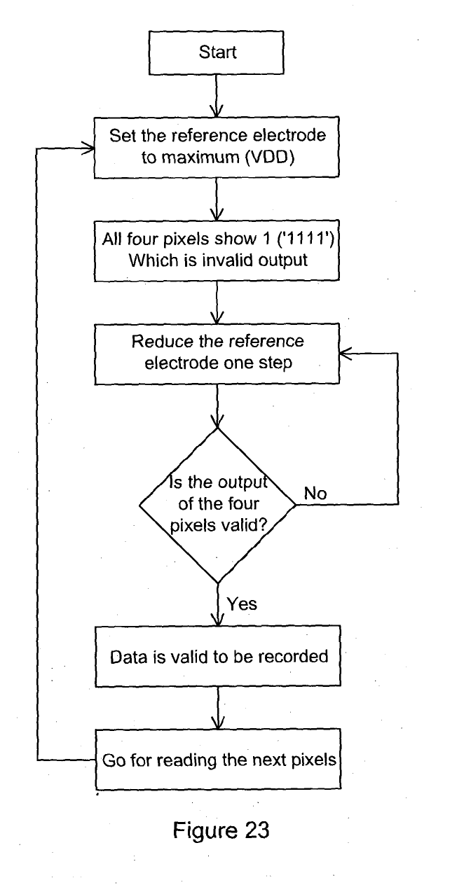

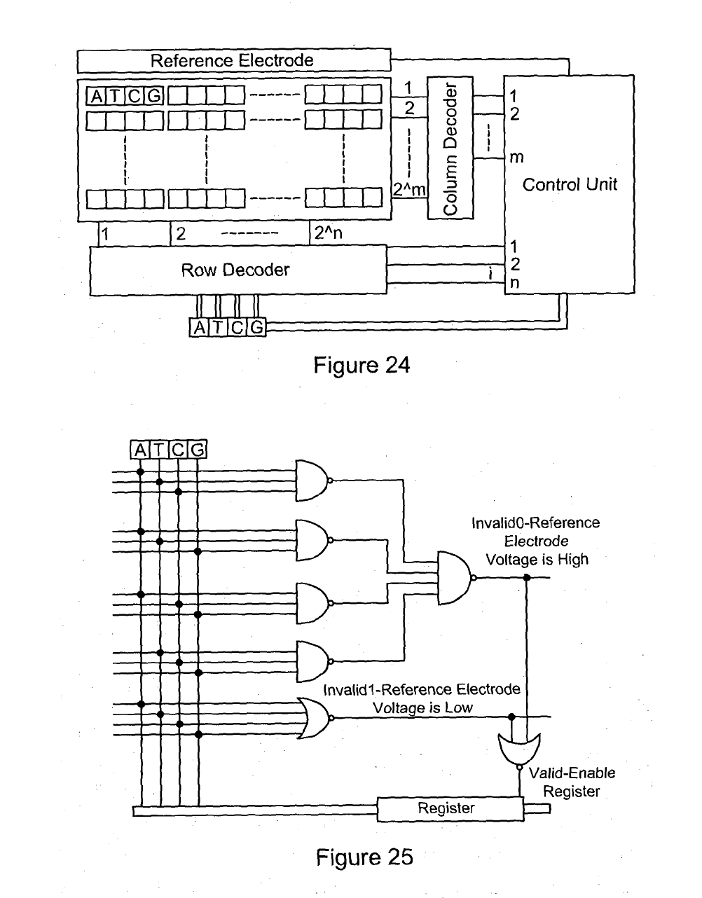

The REFET-less system described earlier can be applied to the DNAGA as well. Each set of four chambers (i.e. those that are being used to detect the polymorphisms of the same gene using the four nucleotides that potentially can extend a common primer) indicate when an error happens and what the outcome is. This method once again benefits from the same advantages, including increased reliability and degree of freedom in designing the reference electrode, in addition to simplifying the processing and control unit, and also reducing the memory and power required. FIG. 24 shows an example of a REFET-less DNAGA architecture. The data can now be read when required and it doesn't necessarily need to be monitored. The same method of adjustment described in the previous paragraph can be used in this configuration (adjusting the reference electrode voltage in case of error). In the same way that the previous REFET-less system had a truth table, Table 2 shows the truth table for a REFET-less DNAGA, and FIG. 23 is a flow diagram that outlines the process (or algorithm) of calibrating the REFET-less DNAGA by adjusting the voltage of the reference electrode and identifying when to record the data.

TABLE 2

| ACTG | |
|---|---|
| Tubes Output | Result |
| 0000 | AR (Adjustment Required) |
| 0001 | Homozygote with type G |
| 0010 | Homozygote with type T |
| 0011 | Heterozygote with type TG |
| 0100 | Homozygote with type C |
| 0101 | Heterozygote with type CG |
| 0110 | Heterozygote with type CT |
| 0111 | AR |
| 1000 | Homozygote with type A |
| 1001 | Heterozygote with type AG |
| 1010 | Heterozygote with type AT |
| 1011 | AR |
| 1100 | Heterozygote with type AC |
| 1101 | AR |
| 1110 | AR |
| 1111 | AR |

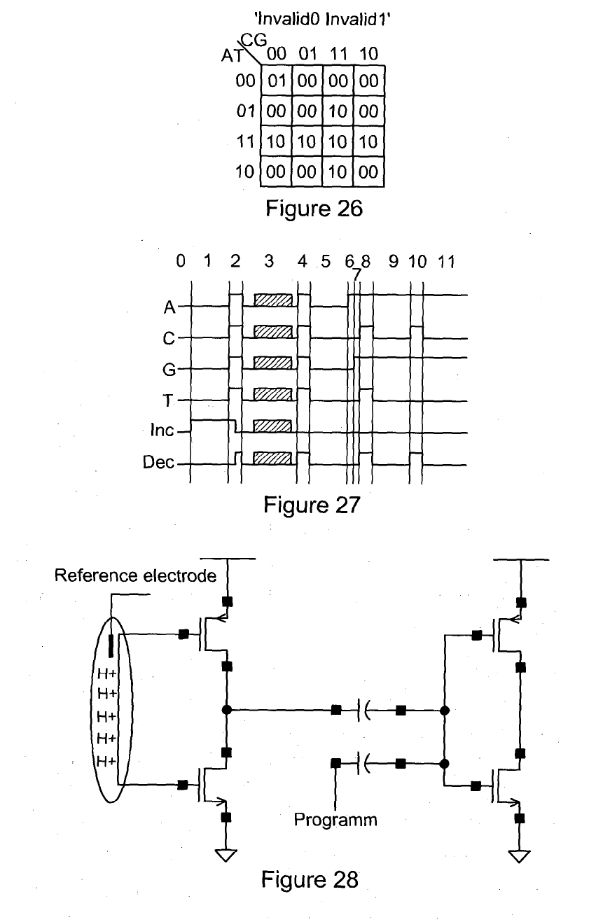

FIG. 25 shows an example of the implementation of the combinational logic for detection of invalid states in REFET-less DNAGA, and FIG. 26 shows a sample Karnaugh-map for error detection logic implemented in FIG. 25. “Invalid0” detects when the reference electrode voltage is too high and “Invalid1” detects when it is too low.

### A Sample Scenario

A sample scenario will now be described to exemplify how such a system can work. The adjustment can take place in two ways, i.e. by increasing and decreasing the reference voltage. FIG. 27 represents the adjustment and detection of SNPs for a heterozygote case according to the sample scenario.

In phase 0, the start of the test (it can even be the start of reading sometime after the start of the test, it works for both cases), the reference electrode voltage is low and all the pixels give an output of 0. Therefore the increment signal (Inc) is increased to 1 and the reference electrode voltage increases in phase 1 until all the pixels switch to 1. Entering phase 2, Inc becomes zero and the decrement signal (Dec) becomes 1 resulting in a decrease in the reference electrode voltage one step until all the pixels give 0. Now, in phase 3, the system is calibrated and all that is required is to wait for the process to finish. During this time the pixels' output can be read and calibration/adjustment can continue if required. For example, as shown in phase 4, an error can occur that arises due to a non-ideal signal detected by all the pixels causing them to switch to 1. The decrement signal (Dec) turns on and reduces the reference electrode voltage so that they all switch back to 0. In this case the unwanted erroneous offset is cancelled out and the system returns to a calibrated phase 5.

In phase 6, due to a pH change in pixel A, the output of pixel A switches to 1. Then the pH in G also drops at phase 7. Now the SNPs have been detected. However, at the time of reading we might face an error such as in phase 8 that turns the other pixels on as well. The error is once again removed by adjusting the reference electrode. In phase 9 the data is ready.

It is possible that an error occurs in one pixel and not in another one. Such a scenario can be remedied as shown in phase 10, where again the reference electrode is decreased for adjustment.

The method of incrementing till switching and then moving backward one step can be done using two types of Major and Minor steps. This can start from the beginning of the experiment or it can be run at the time that the procedures are expected to be finished and the result is available.

The situation for a DNAGA with a REFET is also similar to the REFET-less scenario, except that the increment and decrement direction for adjusting the reference electrode voltage takes place only according to the switching of the Reference pixel and the data is valid only when a working pixel switches when the Reference pixel is 0.

### Another Pixel Configuration

The main concept and methodology has now been discussed. In the adjustment procedures previously discussed, the reference electrode was targeted. Another possibility is to use a programmable inverter to adjust its switching threshold. This is illustrated in FIG. 28, which shows an inverter stack configuration with the second inverter being programmable for adjustment of the switching point.

The reason for the adjustment to be applied to the stacked inverter is that doing that work with the ISFET reduces the fraction of the chemical voltage seen by the floating gate even more. Also, in the situation where the technology does not allow for the stacked inverter being a programmable inverter due to the large capacitance relative to the MOS input capacitance used for programming (large for the ISFET as the fan-in cannot support fan-out), another stacked inverter can be used to adjust the load for the ISFET too. However, this is a matter of technology specifications and designer compromises.

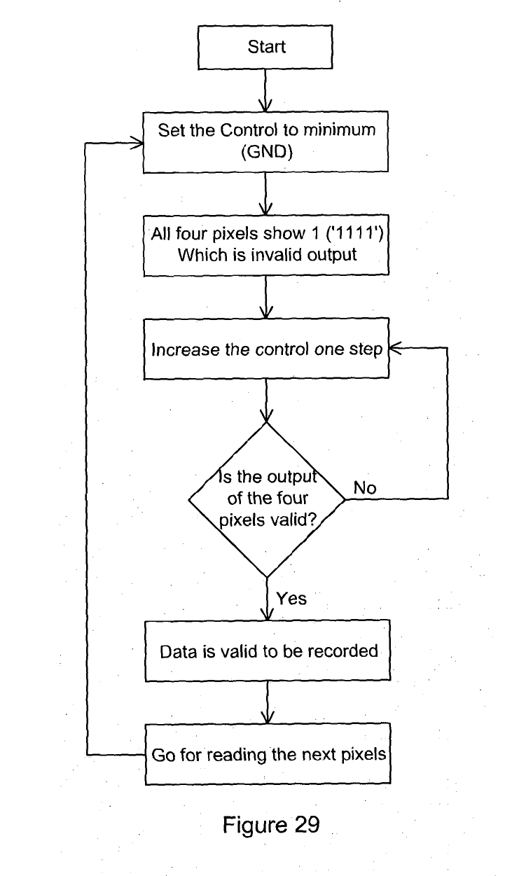

FIG. 29 is a flow diagram that outlines the process (or algorithm) of calibrating the REFET-less DNAGA when a programmable gate inverter is used and identifying when to record the data.

### Summary

Novel methods have been discussed that overcome the difficulties that are experienced with current ISFET-based sequencing and SNP detection chips, in particular power and memory. The complexity of the current chips is required in order to deal with the non-ideal signals and behaviours, both chemical and electronic. They require high precision ADCs and need large amounts memory for recording and processing the data they measure and collect.

Two novel configurations for ISFET-based SNP detection have been described that are not as memory and power intensive as the known architectures, and that do not need the same level of complexity for processing and calibration. They can be calibrated relatively when a reading is required while cancelling out the non-ideal signals. In the first method described, a differential configuration is configured in such a way that no REFET is required. This increases the reliability because of the interstitial comparison that is made (i.e. the comparison of signals from different chambers). The offset is eliminated when it affects the output and it is removed by the adjustment in the biasing (reference electrode or the current source of differential pair). The output can be digitized by replacing the differential amplifier with a comparator, or it can be converted to a digital signal with a less precise converter as the signals are already differential and do not need to be recorded with a high precision and high sampling rate. The data can be read and monitored at the time it is required. There is no need to follow a behaviour and track all the time.

In the second approach, the signal is amplified so that the output is already digital: it is either 1 (vdd) or 0 (gnd). Errors are detected based on invalid outcomes while a recursive method of adjustment and calibration is used. The adjustment is suggested to be made at the reference electrode or the programmable gate of a stacked inverter. This also provides simplicity as the results are compared with each other and moreover the calibration can be done at the time of reading.

The concepts proposed here provide a higher degree of freedom in designing SNP detection arrays, especially with respect to the design of the reference electrode as these are not required to be the same for the whole array but instead are only require the same voltage/potential for each set of four chambers. Reliability can be increased and the chip design made much easier. As a consequence, the need for memory and power has been reduced, and the data can be further read and processed, as may be required by the assessment and/or research that is being carried out, using combinational logic gates. The gates are now driven by the DNA strand extensions and the calibration, readout and process can be done at the same time as reading data, with no need for monitoring and interpreting a behaviour. The methods and configurations described facilitate the design of ISFET-base SNP detection arrays particularly in terms of power, memory, design of reference electrode, critical dependency on a single REFET, wiring and routing. There is no need for high precision ADCs and the data can be collected at the time needed so that it is not necessary to monitor the reactions. The redundant data is not accumulated and only single bits of information are read.

It will be appreciated by the person of skill in the art that various modifications may be made to the above described methods and embodiments without departing from the scope of the present invention.

### REFERENCES

[1] D. M. Garner, Hua Bai, P. Georgiou, T. G. Constandinou, S. Reed, L. M. Shepherd, W. Wong, K. T. Lim and C. Toumazou, “A multichannel DNA SoC for rapid point-of-care gene detection,” in Solid-State Circuits Conference Digest of Technical Papers (ISSCC), 2010 IEEE International, 2010, pp. 492-493.
[2] H. Fletcher, I. Hickey and P. Winter, BIOS Instant Notes in Genetics. Taylor & Francis, 2007.
[3] S. Purushothaman, “The Application of Ion Sensitive Field Effect Transistor Technology to DNA Sequencing,” 2006.
[4] P. Bergveld, “Thirty years of ISFETOLOGY: What happened in the past 30 years and what may happen in the next 30 years,” Sensors Actuators B: Chem., vol. 88, pp. 1-20, 1/1, 2003.
[5] J. Bausells, J. Carrabina, A. Errachid and A. Merlos, “Ion-sensitive field-effect transistors fabricated in a commercial CMOS technology,” Sensors Actuators B: Chem., vol. 57, pp. 56-62, 9/7, 1999.
[6] B. Razavi, Design of Analog CMOS Integrated Circuits. McGraw-Hill, 2001.
[7] J. M. Rothberg, “Integrated Sensor Arrays for Biological and Chemical Analysis,”, 2011
[8] L. M. Shepherd, “Low-power Computational Interfacing with CMOS ISFETs,” 2008.
[9] P. Georgiou, “Chemical Bionics—A Novel Design Approach using Ion Sensitive Field Effect Transistors,” 2008.
[10] P. A. Hammond, D. Ali and D. R. S. Cumming, “Design of a single-chip pH sensor using a conventional 0.6-&mu;m CMOS process,” Sensors Journal, IEEE, vol. 4, pp. 706-712, 2004.
[11] S. Purushothaman, C. Toumazou and J. Georgiou, “Towards fast solid state DNA sequencing,” in Circuits and Systems, 2002. ISCAS 2002. IEEE International Symposium on, 2002, pp. IV-169-IV-172 vol. 4.
[12] S. Purushothaman, C. Toumazou and C. Ou, “Protons and single nucleotide polymorphism detection: A simple use for the Ion Sensitive Field Effect Transistor,” Sensors Actuators B: Chem., vol. 114, pp. 964-968, 4/26, 2006.
[13] C. Toumazou and S. Purushothaman, “Sensing Apparatus and Method,” US 2010/0151479, 2010.
[14] P. Georgiou and C. Toumazou, “ISFET characteristics in CMOS and their application to weak inversion operation,” Sensors Actuators B: Chem., vol. 143, pp. 211-217, 12/4, 2009.
[15] W. Wong, L. Shepherd, P. Georgiou and C. Toumazou, “Towards ISFET based DNA logic for rapid nucleic acid detection,” in Sensors, 2009 IEEE, 2009, pp. 1451-1454.
[16] J. M. Rothberg, “An integrated semiconductor device enabling non-optical genome sequencing,” Nature, vol. 475, pp. 348, 2011.
[17] A. Al-Ahdal and C. Toumazou, “ISFET Based Chemical Switch,” Sensors Journal, IEEE, vol. PP, pp. 1-1, 2011.

1. Apparatus comprising a module for detecting a single nucleotide polymorphism, SNP, within a genome and comprising:
four reaction chambers for receiving a fluid sample, the reaction chambers each containing a different, single one of the bases A, C, G, T, and an identical primer;
a reference electrode which in use is immersed in said fluid sample;
a pair of Ion Sensitive Field Effect Transistors, ISFETs, within each reaction chamber, the ISFETs of each pair comprising respective sensing membranes, or a common sensing membrane, exposed within the associated reaction chamber; and
a plurality of difference detectors, each difference detector having a pair of inputs coupled respectively to outputs of ISFETs associated with different reaction chambers.

2. Apparatus according to claim 1, said plurality of difference detectors each having pairs of inputs coupled to ISFET outputs of different pairs of reaction chambers.

3. Apparatus according to claim 2 and comprising four difference detectors X, Y, Z and W, these having inputs coupled to reaction chamber ISFETs as follows:
X: A and T
Y: T and C
Z: C and G
W: A and G.

4. Apparatus according to claim 1 and comprising an output detector for monitoring the outputs provided by the difference detectors in order to detect output states indicative of nucleotide additions that are not possible.

5. Apparatus according to claim 4 and comprising a processor coupled to said output detector and configured to react to detection of a not possible state by performing an adjustment of the apparatus to eliminate said not possible state.

6. Apparatus according to claim 5, said adjustment being made to the offset of the plurality of difference detectors.

7. Apparatus according to claim 6, said processor being configured to perform said adjustment by one or a combination of: altering a bias voltage applied to said reference electrode, altering the difference detector bias current, altering a bias reference current, and altering the current through a buffer stage.

8. Apparatus according to claim 1, wherein the difference detectors are differential amplifiers.

9. Apparatus according to claim 1, wherein the difference detectors are trans-conductance amplifiers.

10. Apparatus according to claim 1, wherein the difference detectors are comparators.

11. Apparatus according to claim 1 and comprising a plurality of said modules configured to detect different SNPs.

12. A method of detecting a Single Nucleotide Polymorphism, SNP, within a genome, the method comprising:
receiving a fluid sample containing a genome in four reaction chambers, the reaction chambers each containing a different, single one of the bases A, C, G, T, and an identical primer;
detecting any pH changes within the reaction chambers using a pair of Ion Sensitive Field Effect Transistors, ISFETs, within each reaction chamber, the ISFETs of each pair comprising respective sensing membranes, or a common sensing membrane, exposed within the associated reaction chamber; and
providing an output from a plurality of difference detectors, each difference detector having a pair of inputs coupled respectively to outputs of the ISFETs associated with different reaction chambers.

13. A method according to claim 12, the method further comprising monitoring the outputs provided by the difference detectors in order to detect output states indicative of nucleotide additions that are not possible.

14. A method according to claim 13, wherein monitoring the outputs provided by the difference detectors comprises looking up the outputs in a truth table.

15. A method according to claim 13, further comprising reacting to detection of a not possible state by performing an adjustment of the apparatus to eliminate said not possible state.

16. A method according to claim 15, wherein said adjustment is made to the offset of the plurality of difference detectors.

17. A method according to claim 16, wherein performing an adjustment of the apparatus comprises one or a combination of: altering a bias voltage applied to a reference electrode, altering the difference detector bias current, altering a bias reference current, and altering the current through a buffer stage.

18. A method according to claim 12, wherein the difference detectors are differential amplifiers.

19. A method according to claim 12, wherein the difference detectors are trans-conductance amplifiers.

20. A method according to claim 12, wherein the difference detectors are comparators.

21. Apparatus comprising a module for detecting a single nucleotide polymorphism, SNP, within a genome and comprising:
four reaction chambers for receiving a fluid sample, the reaction chambers each containing a different one of the bases A, C, G, T, and a primer;
a reference electrode which in use is immersed in said fluid sample; and
associated with each reaction chamber
a pair of Ion Sensitive Field Effect Transistors, ISFETs, the ISFETs of each pair comprising respective sensing membranes, or a common sensing membrane, exposed within the associated reaction chamber, and the pair of ISFETs being coupled in a push-pull configuration;
a MOSFET inverter stage having an input coupled to an output of said push-pull configuration; and
an output stage providing a digital output that is switchable in dependence upon the voltage present at said sensing membrane(s).

22. Apparatus according to claim 21, said MOSFET inverter stage comprising one MOSFET inverter, or a number of stacked MOSFET inverters.

23. Apparatus according to claim 22, comprising a reference chamber, the reference chamber containing a non-matching primer.

24. Apparatus according to claim 23 and comprising a reference output detector for monitoring the output of the reference chamber in order to detect erroneous deviation within the apparatus.

25. Apparatus according to claim 24 and comprising a processor coupled to said reference output detector and configured to react to detection of erroneous deviation by performing an adjustment of the apparatus to eliminate said deviation.

26. Apparatus according to claim 21, and comprising an output detector for monitoring the digital outputs of the output stage in order to detect output states indicative of nucleotide additions that are not possible.

27. Apparatus according to claim 26, and comprising a processor coupled to said output detector and configured to react to detection of a not possible state by performing an adjustment of the apparatus to eliminate said not possible state.

28. Apparatus according to claim 25, said processor being configured to perform said adjustment by altering a bias voltage applied to said reference electrode.

29. Apparatus according to claim 25, said processor being configured to perform said adjustment by altering a programmable inverter to adjust a switching threshold.

30. Apparatus according to claim 21, and comprising a plurality of said modules configured to detect different SNPs.

31. A method of detecting a Single Nucleotide Polymorphism, SNP, within a genome, the method comprising:
receiving a fluid sample in four reaction chambers, the reaction chambers each containing a different one of the bases A, C, G, T, and a primer;
detecting any pH changes within the reaction chambers using a pair of Ion Sensitive Field Effect Transistors, ISFETs, associated with each reaction chamber, the ISFETs of each pair comprising respective sensing membranes, or a common sensing membrane, exposed within the associated reaction chamber, and the pair of ISFETs being coupled in a push-pull configuration;
coupling the output of the push-pull ISFETs to the input of a MOSFET inverter stage; and
providing a digital output from an output stage, the digital output being switchable in dependence upon the voltage present at said sensing membrane(s).

32. A method according to claim 31, comprising also performing the steps of the method on a reference chamber, the reference chamber containing a non-matching primer.

33. A method according to claim 32, comprising monitoring the output of the reference chamber in order to detect erroneous deviation within the apparatus.

34. A method according to claim 33, comprising reacting to the detection of erroneous deviation by performing an adjustment of the apparatus to eliminate said deviation.

35. A method according to claim 31, comprising monitoring the digital outputs of the output stage in order to detect output states indicative of nucleotide additions that are not possible.

36. A method according to claim 35, wherein monitoring the digital outputs provided by the output stage comprises looking up the outputs in a truth table.

37. A method according to claim 35, comprising reacting to the detection of a not possible state by performing an adjustment of the apparatus to eliminate said not possible state.

38. A method according to claim 34, wherein performing said adjustment comprises altering a bias voltage applied to a reference electrode.

39. A method according to claim 34, wherein performing said adjustment comprises altering a programmable inverter to adjust a switching threshold.
